(19) United States
(12) Patent Application Publication
(10) Pub. No.: US 2008/0287373 A1
(43) Pub. Date: Nov. 20, 2008

(54) Topical skin treating kits

(76) Inventor: Karl F. Popp, Schodack Landing, NY (US)

Correspondence Address: NATH & ASSOCIATES, 112 South West Street, Alexandria, VA, 22314, US

(21) Appl. No.: 11/798,922

(22) Filed: May 17, 2007

(51) Int. Cl. A61K 31/70; A61P 17/00; A61P 17/10

(52) U.S. Cl. 514/25

(57) ABSTRACT

A kit comprising a soap-free cleanser and a topical composition which comprises clindamycin or a pharmaceutically acceptable salt or ester thereof, and a pharmaceutically acceptable carrier, formulations and packages thereof, and methods of using the same to treat various skin disorders.

### FIELD OF THE INVENTION

The present subject matter relates generally to a kit comprising a soap-free cleanser and a topical composition which comprises clindamycin or a pharmaceutically acceptable salt or ester thereof, and a pharmaceutically acceptable carrier, formulations and packages thereof, and methods of using the same to treat various skin disorders.

### BACKGROUND OF THE INVENTION

Skin disorders involving the sebaceous glands and follicles in humans include conditions such as acne and rosacea, as well as other noninfectious dermatological diseases involving microorganisms. Such disorders are often marked by inflammation.

Acne is a condition of the human skin characterized by an excess flow of sebum, or skin oil, from the sebaceous glands located in the pilosebaceous apparatus. Sebum reaches the skin surface through the duct of the hair follicle. The presence of excessive amounts of sebum in the duct and on the skin acts to block or stagnate the continuous flow of sebum from the follicular duct, thus producing a thickening and a solidification of the sebum to form a solid plug known as a comedone. When this process occurs, hyperkeratinization of the follicular opening is stimulated, thus completely closing the duct. The usual results are papules, pustules, or cysts, often contaminated with bacteria which cause secondary infections. Acne is particularly characterized by the presence of comedones, inflammatory papules, pustules, or cysts. The effect of acne ranges from slight skin irritation and pitting to disfiguring scars.

In the past, these dermatological disorders have been treated with oral and/or topical antibacterial agents. The oral antibiotics used include tetracycline, erythromycin, and minocycline. The topical compositions used have separately contained the antibiotics tetracycline, erythromycin, and clindamycin; retinoids such as retinoic acid or tretinoin; and benzoyl peroxide, which exerts its antibacterial action via its potent oxidizing properties.

One currently available product, Cleocin T® brand clindamycin topical solution by Pharmacia & Upjohn Company of Kalamazoo, Mich., is a topical solution containing 1% of clindamycin. Cleocin T®, however, has several drawbacks. For one, the formulation contains 50% isopropyl alcohol and water. This formulation often proves to be excessively drying and irritating to the skin.

However, many of the presently known topical compositions for the treatment of acne are formulated for administration to patients twice per day. It has been reported that patient compliance with compositions that must be administered twice per day tends to be irregular, especially among teenagers who are the primary sufferers of acne.

Further, the current treatment options pose a significant risk of adverse side effects. For example, clindamycin, which is well absorbed through the skin, has been associated with colitis, diarrhea, and bloody diarrhea. Severe colitis may result in death. Accordingly, there is a need to reduce the potential side effects of these prior compositions by reducing the number of required daily exposures to them.

Accordingly, there remains a need in the art for topical treatments for treating a dermatological disorder that are capable of treating an affected area of skin while reducing the irritation associated with the topical composition. In addition, there remains a need in the art for enhancing the efficacy of such a treatment of an area of skin afflicted with acne. The present subject matter addresses these needs.

### SUMMARY OF THE INVENTION

The present subject matter relates generally to methods and kits for reducing irritation associated with a clindamycin treatment. The present subject matter also relates generally to a method for enhancing the efficiency of a clindamycin treatment.

In this regard, a preferred embodiment of the present subject matter relates to a kit comprising a soap-free cleanser and a topical composition which comprises clindamycin or a pharmaceutically acceptable salt or ester thereof, and a pharmaceutically acceptable carrier.

In a further preferred embodiment, the present subject matter also relates to a package comprising: an outer container; and a first product container and a second product container within the outer container, wherein the first product container contains a soap-free cleanser; and the second product container contains a topical composition which comprises clindamycin or a pharmaceutically acceptable salt or ester thereof, and a pharmaceutically acceptable carrier.

In another preferred embodiment, the present subject matter relates to a method for treating a skin disorder or condition in a patient, comprising: topically administering a soap-free cleanser to skin of a patient in need therof; removing the soap-free cleanser from the skin; topically administering to the patient a topical composition which comprises clindamycin or a pharmaceutically acceptable salt or ester thereof, and a pharmaceutically acceptable carrier in an amount effective to treat the skin disorder or condition.

### DETAILED DESCRIPTION OF THE INVENTION

### Definitions

As used herein, “derivative” or “derivatives” refers to derivative(s) of the active compound(s) which possess the same pharmacological activity as the active compound(s) and which are neither biologically nor otherwise undesirable. Derivatives of the active compounds include, without limitation, polymorphs, solvates, salts, N-oxides, hydrates, dehydrates, crystalline forms, anhydrous forms, amorphous forms, and mixtures thereof.

As used herein, an “extended period of time” refers to the shelf life of the presently preferred compositions, including time spent on the shelf at a pharmacy as well as the entire time period after sale of the composition during which the composition remains effective for the indicated use.

As used herein, a “patient” refers to an entity to whom the preferred drug compositions are being administered. Non-limiting examples of a patient in this regard include a mammal, an animal and a human being. Preferably, the patient is a human being.

As used herein, “pharmaceutically acceptable salts” or “salts” refers to salts of the active compound(s) which possess the same pharmacological activity as the active compound(s) and which are neither biologically nor otherwise undesirable. A salt can be formed with, for example, organic or inorganic acids. Non-limiting examples of suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, naturally and synthetically derived amino acids.

Non-limiting examples of base salts include ammonium salts; alkali metal salts, such as sodium and potassium salts; alkaline earth metal salts, such as calcium and magnesium salts; salts with organic bases, such as dicyclohexylamine salts; methyl-D-glucamine; and salts with amino acids, such as arginine, lysine, and so forth. Also, the basic nitrogen-containing groups can be quaternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; asthma halides, such as benzyl and phenethyl bromides; and others. Water or oil-soluble or dispersible products are thereby obtained. Preferred salts include acetate, butyrate, hemisuccinate and phosphate.

As used herein, the terms “administering”, “administration”, and like terms refer to any method which, in sound medical or cosmetic practice, delivers the composition to a subject in such a manner as to provide a positive effect on a dermatological disorder, condition, or appearance. The compositions are preferably administered such that they cover the entire area to be treated. “Direct administration” refers to any method which, in sound medical or cosmetic practice, delivers the composition to a subject without the use of another composition, delivery agent, or device. “Indirect administration” refers to any method which, in sound medical or cosmetic practice, delivers the composition to a subject with the use of at least one other composition, delivery agent, or device.

As used herein, the phrase “commercial purposes” refers to any purposes requiring any length of time or storage condition in accordance with FDA rules or regulations, including shipping time, storage, distribution, and refrigeration.

As used herein, the phrases an “effective amount” or a “therapeutically effective amount” of an active agent or ingredient, or pharmaceutically active agent or ingredient, which are synonymous herein, refer to an amount of the pharmaceutically active agent sufficient enough to have a positive effect on the area of application. Accordingly, these amounts are sufficient to modify the skin disorder, condition, or appearance to be treated but low enough to avoid serious side effects, within the scope of sound medical or dermatological advice. A therapeutically effective amount of the pharmaceutically active agent will cause a substantial relief of symptoms when applied repeatedly over time. Effective amounts of the pharmaceutically active agent will vary with the particular condition or conditions being treated, the severity of the condition, the duration of the treatment, the specific components of the composition being used, and like factors.

As used herein, “storage stable” refers to the ability of the present compositions to have a long shelf life, including time spent on the shelf at a pharmacy as well as the entire time period after sale of the composition, during which time the composition maintains its effectiveness and pharmaceutically acceptable appearance. Accordingly, the present compositions are stable in that they exhibit a minimum amount of degradation during an extended period of storage.

The term “sensitivity” refers to the degree of skin irritation or skin inflammation, as exemplified by parameters in suitable assays for measuring sensitivity, inflammation, irritation, and the like. One such assay is the Jordan-King assay.

The term “acne” means a common inflammatory disease of the pilosebaceous glands characterized by comedones, papules, pustules, inflamed nodules, superficial pus-filled cysts, and (in extreme cases) canalizing and deep, inflamed, sometimes purulent sacs. Types of acne within the scope of the present inventive subject matter include acne vulgaris or topical acne. “Acne” is caused by an interaction among hormones, keratin, sebum, and bacteria. One common bacterial causative agent is Propionibacterium acnes.

As used herein, “short-contact therapy” and “short-contact therapies” refer to compositions herein, such as, for example, the soap-free cleanser, which are formulated and intended to be in contact with the skin for a limited amount of time. As a short-contact therapy, it is preferable these compositions are removed from the affected area after a time of about 15 seconds to about 30 minutes. In a preferred example, the soap-free cleanser is removed after a time of about 15 seconds to about 120 seconds. In a more preferred example, the soap-free cleanser is removed after a time of about 15 seconds to about 60 seconds. Optimally, the short contact therapy is removed from the skin using water.

The term “soap” refers to a substance used for washing and cleansing purposes, usually made by treating a fat with an alkali, such as sodium or potassium hydroxide, and consisting chiefly of the sodium or potassium salts of the acids contained in the fat.

The term “synthetic detergent” refers to a non-soap detergent. Thus, a synthetic detergent is not an alkali fatty acid product.

As used herein, the terms “non-soap” and “soap-free” are used interchangeably and refer to a composition or compositions that do not contain soap.

As used herein, “affected area” refers to the area of skin afflicted with acne on a patient. As used herein, a “treatment” or “treating” of a skin disease, disorder, or condition encompasses alleviation of at least one symptom thereof, a reduction in the severity thereof, or the delay, prevention, or inhibition of the progression thereof. Treatment need not mean that the disease, disorder, or condition is totally cured. A useful composition herein needs only to reduce the severity of a skin disease, disorder, or condition, reduce the severity of symptoms associated therewith, provide improvement to a patient's quality of life, or delay, prevent, or inhibit the onset of a skin disease, disorder, or condition.

Other terms as used herein are meant to be defined by their well-known meanings in the art.

### Kits

The present subject matter is directed to a kit, comprising:

a soap-free cleanser; and

a topical composition which comprises clindamycin or a pharmaceutically acceptable salt or ester thereof, and a pharmaceutically acceptable carrier.

### Soap-Free Cleanser

The present kits include a soap free cleanser. The soap free cleanser preferably comprises water, at least one emollient, a surfactant, and at least one preservative.

The soap-free cleanser of the present subject matter typically contains water as a main component. The amount of water present in the soap-free cleanser preferably is from about 85% by weight to about 95% by weight of the soap-free cleanser. More preferably, the amount of water present in the soap-free cleanser is from about 88% by weight to about 92% by weight of the soap-free cleanser. Most preferably, the amount of water present in the soap-free cleanser is from about 90% by weight to about 92% by weight of the soap-free cleanser.

The soap-free cleanser of the present subject matter also preferably comprises at least one emollient. Preferably, the soap-free cleanser comprises a mixture of two or more emollients. Emollients are components which may soften or soothe the skin. Thus, the emollients present in the soap-free cleanser may aid in reducing any irritation associated with the topical composition, as well as enhancing the efficacy of the topical composition.

Emollients useful in the soap-free cleansers of the present subject matter include, without limitation, vegetable oils, coconut oil, palm glycerides, olea europaea, extracts thereof, derivatives thereof, and mixtures thereof. Other non-limiting examples of specific emollients useful in the present soap-free cleansers include glycerin, pentylene glycol, butylene glycol, polyethylene glycol, sodium pyrrolidone carboxylate, α-hydroxy acids, β-hydroxy acids, polyhydric alcohols, including stearyl alcohol, ethoxylated and propoxylated polyols, polyols, polysaccharides, panthenol, hexylene glycol, propylene glycol, dipropylene glycol, sorbitol and mixtures thereof. In a preferred embodiment, the at least one emollient is polyethelyene glycol, stearyl alcohol, or a mixture thereof.

The amount of the at least one emollient present in the soap-free cleanser is typically from about 2.5% weight to about 10% by weight of the soap-free cleanser. Preferably, the at least one emollient or combination of emollients is present at an amount of from about 5% by weight to about 10% by weight of the soap-free cleanser. More preferably, the emollients are present in an amount of about 7.5% by weight of the soap-free cleanser.

The soap-free cleanser of the present subject matter may also further comprise a surfactant. The surfactant may aid in the removal of oils and other organic materials from the affected area when the soap-free cleanser is administered thereto.

Classes of surfactants useful in the present subject matter include, without limitation, betaines, amine oxides, amphoteric surfactants, sulfates, isethionates, sulfoacetates, sarcosinates, phosphates, and mixtures thereof. Specific surfactants useful in the soap-free cleanser of the present subject matter include, without limitation, sodium lauryl sulfate, sodium laureth sulfate, ammonium lauryl sulfate, ammonium laureth sulfate, disodium laureth sulfosuccinate, disodium ricinoleamido monoethanolamide sulfosuccinate, sodium cocoyl isethionate, sodium methyl oleoyl taurate, sodium methyl cocoyl taurate, sodium laureth-13 carboxylate, sodium C₁₄₋₁₆ oefin sulfonate, sodium laureth-4 phosphate, laureth-3 phosphate, triethylanolamine lauryl sulfate, magnesium lauryl sulfate, sodium tridecyl sulfate, alpha-olefin sulfate, and mixtures thereof. In a preferred embodiment, the surfactant is sodium cocoyl isethionate.

The surfactant is preferably present in the soap-free cleanser in an amount of about 0.25% by weight to about 2% by weight of the soap-free cleanser. More preferably, the amount of surfactant is from about 0.45% by weight to about 1.5% by weight of the soap-free cleanser. Most preferably, the amount of surfactant is about 0.7% by weight of the soap-free cleanser.

The soap-free cleanser may also include at least one preservative. The presence of at least one preservative may provide sufficient preservative activity adequate to minimize and manage the risk of microbial contamination during storage or use of the soap-free cleanser. In other words, the at least one preservative present in the soap-free cleanser can provide the soap-free cleanser with a long shelf-life. Preferably, more than one preservative is present in the soap-free cleanser.

Preservatives useful in the soap-free cleanser include, without limitation, propylene glycol, glycerol, butylene glycol, pentylene glycol, hexylene glycol, sorbitol, benzyl alcohol, ethanol, methylparaben, propylparaben, butylparaben, derivatives thereof, and mixtures thereof. In a preferred embodiment, the preservative is methylparaben, propylparaben, butylparaben, or a mixture thereof.

The preservatives are preferably present in the soap-free cleanser in a total amount of about 0.1% by weight to about 0.5% by weight of the soap-free cleanser. More preferably, the preservatives are present in a total amount of about 0.3% by weight of the soap-free cleanser.

### Topical Composition

The kits disclosed herein additionally comprise a topical composition. In a preferred embodiment, the topical composition comprises clindamycin or a pharmaceutically acceptable salt or ester thereof and a pharmaceutically active carrier.

The clindamycin component of the present topical compositions is preferably a pharmaceutical grade salt or ester of clindamycin.

Clindamycin phosphate (ester) and clindamycin hydrochloride (salt) are preferred pharmaceutically acceptable salts and esters of clindamycin which can be used in the present composition due to their compatibility with gelling agents and extensive history of topical use.

The clindamycin component of the present topical compositions is generally present at an amount of from about 0.9% to about 2.5% by weight of the total composition. In a preferred embodiment, the present topical compositions contain between about 0.5% and about 1.5% by weight of the topical composition of clindamycin. In a particularly preferred embodiment, the present topical compositions contain about 1.2% by weight clindamycin. The present compositions are unique in that they can be produced having a standard deviation of clindamycin present within ±0.015.

Additionally, the present topical compositions are capable of effectively maintaining a clindamycin composition having not more than 0.02% by weight of clindamycin degradates.

The topical composition may take the form of one or more of a gel, cream, lotion, suspension, emulsion, ointment, or foam. A gel is most preferred in this regard. Other cosmetic treatment compositions known to those skilled in the art, including liquids and balms, are additionally contemplated as falling within the scope of the present subject matter.

In a preferred embodiment, the topical composition herein may be in the form of a gel. In this regard, preferred gel topical compositions herein may comprise a pharmaceutically acceptable carrier comprising an inactive ingredient selected from the group consisting of carbomer, disodium monolauryl sulfosuccinate, disodium EDTA, methyl paraben, poloxamer, glycerin, dimethicone, hydrated silica, sodium hydroxide, purified water, and mixtures thereof.

Emulsions, such as oil-in-water or water-in-oil systems, as well as a base (vehicle or carrier) for the topical formulation can be selected to provide effectiveness of the active ingredient and/or avoid allergic and irritating reactions (e.g., contact dermatitis) caused by ingredients of the base or by the active ingredients.

Creams useful in the present compositions may also be semisolid emulsions of oil and water, are easily applied, and can vanish when rubbed into the skin.

In this regard, suitable lotions or creams may contain, for example, a mixture of one or more of the following: mineral oil, sorbitan monostearate, polysorbate 60, cetyl ester wax, de minimis cetearyl alcohol, de minimis 2-octyldodecanol, de minimis benzyl alcohol, and water.

Ointments which may be useful herein are oleaginous and contain little if any water; feel greasy but are generally well tolerated; best used to lubricate, especially if applied over hydrated skin; they are preferred for lesions with thick crusts, lichenification, or heaped-up scales and may be less irritating than cream for some eroded or open lesions (e.g., stasis ulcers). Drugs in ointments are often more potent than in creams.

In this regard, suitable ointments may contain, for example, a mixture of one or more of the following: mineral oil, liquid petrolatum, white petrolatum, propylene glycol, polyoxyethylene polyoxypropylene compound, emulsifying wax, and water.

In a preferred embodiment, the topical composition exhibits a final pH of about 4.5 to about 5. In a particularly preferred embodiment, the topical composition exhibits a final pH of about 4.6 to about 4.8.

### Dermatologically Acceptable Excipients

The topical composition and soap-free cleanser discussed herein can additionally comprise at least one dermatologically acceptable excipient commonly known to those of ordinary skill in the art as useful in topical compositions. Preferred, non-limiting examples of dermatologically acceptable excipients useful in these drug compositions are those selected from the group consisting of preservatives, colorants or pigments, anti-oxidants, radical scavengers, surfactants, emulsifiers, humectants, pH modifiers, chelating agents, derivatives thereof, and mixtures thereof.

### Preservatives

The presently topical compositions may optionally further contain at least one preservative. Preferred non-limiting examples of preservatives useful in this regard include propylene glycol, glycerol, butylene glycol, pentylene glycol, hexylene glycol, sorbitol, benzyl alcohol, ethanol, derivatives thereof, and mixtures thereof.

If present, the preservative is preferably present in an amount of about 0.1% to about 2.5% by weight of the overall weight of the composition.

### Humectants

The present topical compositions may optionally further contain a humectant. Preferred, non-limiting examples of humectants useful in this regard include sorbitol, sorbitol syrup, E965 maltitol, maltitol, maltitol syrup, E1200 polydextrose, E1518 glyceryl triacetate, triacetin, glyceryl triacetate, 1,2,3-propanetriyl triacetate, 1,2,3-propanetriol triacetate, triacetylglycerol, E1520 propylene glycol, 1,2-propanediol, 1,2-dihydroxypropane, methylethylene glycol, propane-1,2-diol, E420 sorbitol, propylene glycol, polyethylene glycol (PEG) esters, PEG-20 stearate, PEG-40 stearate, PEG-150 stearate, PEG-150 distearate, PEG-100 stearate, laureth-12, ceteareth-20, laureth-23, glycereth-7, glycereth-12, glycereth-26, PEG-4, PEG-6, PEG-8, PEG-12, PEG-32, PEG-75, PEG-150, derivatives thereof, and mixtures thereof.

### pH Modifiers

The present compositions may optionally further contain a pH modifier. Preferred non-limiting examples of pH modifiers useful in this regard include inorganic hydroxides, inorganic oxides, inorganic salts of weak acids, inorganic acids, organic acids, derivatives thereof, and mixtures thereof.

Preferred, non-limiting examples of inorganic hydroxides useful in this regard include ammonium hydroxide, alkali metal hydroxide, alkaline earth metal hydroxides, derivatives thereof, and mixtures thereof.

Preferred inorganic hydroxides useful herein include ammonium hydroxide, monovalent alkali metal hydroxides such as sodium hydroxide and potassium hydroxide, divalent alkali earth metal hydroxides such as calcium hydroxide and magnesium hydroxide, derivatives thereof, and mixtures thereof.

Preferred, non-limiting examples of inorganic oxides useful herein include magnesium oxide, calcium oxide, derivatives thereof, and mixtures thereof.

Preferred, non-limiting examples of inorganic salts of weak acids useful herein include ammonium phosphate (dibasic), alkali metal salts of weak acids such as sodium acetate, sodium borate, sodium metaborate, sodium carbonate, sodium bicarbonate, sodium phosphate (tribasic), sodium phosphate (dibasic), potassium carbonate, potassium bicarbonate, potassium citrate, potassium acetate, potassium phosphate (dibasic), potassium phosphate (tribasic), alkaline earth metal salts of weak acids such as magnesium phosphate and calcium phosphate, derivatives thereof, and mixtures thereof.

Preferred, non-limiting examples of inorganic acids useful herein include hydrochloric acid, hydrofluoric acid, hydrobromic acid, nitric acid, nitrous acid, hydrocyanic acid, perchloric acid, chlorous acid, sulfurous acid, hypochlorous acid, phosphoric acid, acetic acid, sulfuric acid, derivatives thereof, and mixtures thereof. Preferred, non-limiting examples of organic acids useful herein include lactic acid, citric acid, glutamic acid, methanoic acid, ethanoic acid, phenol, monochloroethanoic acid, dichloroethanoic acid, trichloroethanoic acid, butanoic acid, salicylic acid, glycolic acid, and mixtures thereof.

Further, mixtures of any of the above-mentioned pH modifiers are also contemplated as within the scope of the present topical composition and soap-free cleanser.

### Chelating Agents

The presently preferred topical composition may optionally further contain a chelating agent. Preferred non-limiting examples of chelating agents useful in this regard include citric acid, isopropyl (mono) citrate, stearyl citrate, lecithin citrate, gluconic acid, tartaric acid, oxalic acid, phosphoric acid, sodium tetrapyrophosphate, potassium monophosphate, sodium hexametaphosphate, calcium hexametaphosphate, sorbitol, glycine (aminoacetic acid), methyl glucamine, triethanolamine (trolamine), EDTA, DEG (dihydroxyethylglycine), DPTA (diethylene triamine pentaacetic acid), NTA (Nitrilotriacetic Acid), HEDTA (N-(hydroxyethyl)-ethylenetriaminetriacetic acid), aminocarboxylates, dimercaperol (BAL), larixinic acid (Maltol), unidentate ligands (fluoride and cyanide ions), diphenylthiocarbazone, 0-phenanthroline, barium diphenylamine sulfonate, sodium glucoheptonate, 8-hydroxyquinoline, olefin complexes (such as dicyclopentadienyl iron), porphyrins, phosponates, pharmaceutically acceptable salts thereof, derivatives thereof, and mixtures thereof.

In addition to those enumerated above, any other anti-inflammatory agent, gelling agent, moisturizer, preservative, colorant or pigment, antioxidant, radical scavenger, surfactant, emulsifier, humectant, pH modifier, chelating agent, or other dermatologically acceptable excipient commonly known to those of ordinary skill in the art as useful in topical compositions are contemplated as useful in the compositions described herein. Further, any non-toxic, inert, and effective topical carrier may be used to formulate the compositions described herein.

Well-known carriers used to formulate other topical therapeutic compositions for administration to humans will be useful in these compositions. Examples of these components that are well known to those of skill in the art are described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, http://www.accessdata.fda.gov/scripts/cder/iig/index.cfm, the contents of which are hereby incorporated by reference in their entirety. Examples of such useful pharmaceutically acceptable excipients, carriers and diluents include distilled water, physiological saline, Ringer's solution, dextrose solution, Hank's solution, and DMSO, which are among those preferred for use herein.

These additional other inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990) and Remington's Pharmaceutical Sciences, 17th Ed., Mack Publishing Co., Easton, Pa. (1990), both of which are incorporated by reference herein in their entirety.

### Additional Active Ingredients

The topical compositions of the instant kit may further comprises at least one additional ingredient selected from the group consisting of benzoyl peroxide, salicylic acid, a retinoid, derivatives thereof, and mixtures thereof.

In this regard, should it be present, the additional benzoyl peroxide component is preferably introduced to the topical composition as a dispersion. The benzoyl peroxide is pharmaceutical grade. The benzoyl peroxide in the dispersion may be in the form of a slurry of a finely divided powder, or in the form of a hydrous granular material which may have its particle size reduced accordingly during processing according to the present inventive subject matter. Preparation of suitable benzoyl peroxide constituents is well described in the medical and patent literature.

The additional benzoyl peroxide component may optionally be added to the topical composition in an amount of between about 1% to about 20% by weight of the total composition. In a preferred embodiment, the topical composition may contain from about 2.25% to about 12.5% by weight of the total composition of benzoyl peroxide. In a particularly preferred embodiment, the present topical compositions may contain about 11% by weight of benzoyl peroxide.

In a final composition according to the present subject matter, the ratio of benzoyl peroxide to clindamycin may be from about 1.8:1 to 12:1. Particularly preferred are compositions wherein the ratio of benzoyl peroxide to clindamycin is from about 4:1 to about 5:1.

Should it be present, the additional retinoid component added to the topical composition is preferably a pharmaceutical grade salt of the retinoid. Pharmaceutically acceptable salts, esters, or derivatives of retinoids refer to those which possess the desired pharmacological activity and which are neither biologically nor otherwise undesirable. The additional retinoid component may optionally be added to the topical composition in an amount of from about 0.01% to about 1.5% by weight of the total composition. In a particularly preferred embodiment, the retinoid is present at an amount of about 0.01% to about 0.5% by weight of the total composition.

Any of a wide variety of retinoids known as useful in treating skin diseases, disorders, or conditions is contemplated as capable of being included in the present compositions. In this regard, preferred non-limiting examples of retinoids useful in the present compositions include tazarotene, retinoic acid, tretinoin, isotretinoin, adapalene, bexarotene, alitretinoin, vitamin A, retinol, retinal, retinyl palmitate, retinyl acetate, ethyl 5-(2-(4,4-dimethylthiochroman-6-yl)ethynyl)thiophene-2-carboxylate, 6-(2-4,4-dimethylthiochroman-6-yl)-ethynyl)-3-pyridylmethanol, 2-(2-(4,4-dimethylthiochroman-6-yl)-ethynyl)-5-pyridinecarboxaldehyde)-5-pyridinecarboxaldehyde, salts therefore, derivatives thereof, and mixtures thereof. Tazarotene, retinoic acid, tretinoin, and isotretinoin, as well as salts or derivatives thereof, are especially preferred in this regard. In a most preferred embodiment, the retinoid is tazarotene or a salt or derivative thereof.

The present topical compositions may be formulated for either once-per-day or twice-per-day administration. In a preferred embodiment, the once-per-day administration is in the evening or at night to increase compliance and to account for skin conditions most favorable to reducing inflammation.

### Combination Therapy

In another preferred embodiment, the present preferred kit may be used in combination with additional pharmaceutical dosage forms to enhance their effectiveness in treating dermatological disorders, particularly acne, described herein. In this regard, the present soap-free cleanser and topical composition may be administered as part of a regimen additionally including any other pharmaceutical and/or pharmaceutical dosage form known in the art as effective for the treatment of acne generally.

Accordingly, the present kits may be used in combination with other compositions containing other active ingredients readily known to those of skill in the art as useful in the topical treatment of acne. Exemplary additional active ingredients include, but are not limited to, macrolide antibiotics, bactericidal drugs, bacteriostatic drugs, cleansing agents, absorbents, anti-infective agents, anti-inflammatory agents, astringents (drying agents that precipitate protein and shrink and contract the skin), emollients (skin softeners), keratolytics (agents that soften, loosen, and facilitate exfoliation of the squamous cells of the epidermis), and mixtures thereof.

Exemplary macrolide antibiotics contemplated in this regard include, but are not limited to, Azithromycin, Clarithromycin, Erythromycin, Lincomycin, and mixtures thereof. The macrolides are similar in structure and activity. All the macrolides are easily absorbed and all are primarily bacteriostatic and bind to the 50S subunit of the ribosome, thus inhibiting bacterial protein synthesis. These drugs are active against aerobic and anaerobic gram-positive cocci, with the exception of enterococci, and against gram-negative anaerobes and useful in the present subject matter.

Exemplary bactericidal drugs (i.e., they kill bacteria) contemplated herein include, but are not limited to, Penicillins, cephalosporins, vancomycin, aminoglycosides, quinolones, and polymyxins.

Exemplary bacteriostatic drugs (i.e., they slow bacterial growth) contemplated herein include, but are not limited to, erythromycin, tetracyclines, chloramphenicol, clindamycin, lincomycin, clarithromycin, azithromycin, and sulfonamides. However, it is well know that some bactericidal drugs may be bacteriostatic against certain microorganisms and vice versa. These drugs are well known in the art and may be found, for example, in The Merck Manual of Diagnosis and Therapy, 13th edition, Section 13, Chapter 153 Anti-bacterial Drugs, 2001, incorporated herein by reference in its entirety.

Furthermore, the soap-free cleanser and topical composition may be used with adjunct therapies and treatments, such as pre-washing with common soaps, and mild detergents. However, selection is important when treating skin disorders such as acne since antibacterial soaps and abrasive soaps may increase irritation and make it difficult to use follicular drugs. Such follicular drugs may include topical antibiotics and antiseptics, as well as intralesional corticosteroids.

In superficial pustular acne, the soap-free cleanser and topical composition may be used in combination with one of the follicular drugs.

Sunlight therapy can be useful in combination with the present subject matter. Sunlight is known to cause mild dryness and slight scaling and is usually helpful. Since sunlight is not always available, some benefit may be obtained with a sunlamp.

Another combination therapy involves Azelaic acid cream 20%, which has antiproliferative and antibacterial effects, and is known to be effective in comedonal or inflammatory acne.

An additional combination therapy contemplated with the present kits is topical tretinoin (retinoic acid) in 0.025%, 0.05%, or 0.1% cream, 0.05% liquid, or 0.01% or 0.025% gel. Also, a new topical retinoid, Differin®) brand adapalene 0.1% gel, Galderma Laboratories, San Antonio, Tex., was recently approved in the USA and may be useful since it may be slightly less irritating than topical tretinoin. Other retinoids which may be useful in combination therapy include Panretin®, containing alitretinoin, and Targretin®, containing bexarotene. Since retinoids must be applied carefully and at night to avoid excessive irritation, a regimen in combination with these drugs may be used over time to achieve results. For example, retinoid therapy may be initiated and then followed on with once a day treatment in accordance with the present subject matter. Exposure to sunlight when using retinoids and concurrent use of other drugs are restricted to prevent severe irritation. However, a back-to-back alternating regimen over a period of weeks or months time may be useful. With tretinoin or adapalene, acne may worsen at first; improvement usually requires ≧3 to 4 weeks.

Other topical drugs include OTC drugs, various sulfur-resorcinol combinations, and oral antibiotics may also be helpful in combination with the present treatment regimen when treating acne.

Similarly, an anti-acne agent other than those specified herein, or an additional topical pharmaceutically active agent, can be added to the present preferred compositions to enhance their effectiveness. Accordingly, this additional agent or additional pharmaceutical dosage form can be applied to a patient either directly or indirectly, and concomitantly or sequentially, with the preferred compositions described herein.

### Package

The present subject matter is further directed to a package comprising:

an outer container; and

a first product container and a second product container within the outer container,

wherein the first product container contains a soap-free cleanser; and

the second product container contains a topical composition which comprises clindamycin or a pharmaceutically acceptable salt or ester thereof, and a pharmaceutically acceptable carrier.

The package of the present subject matter preferably comprises an outer container. The outer container can be any container suitable for holding the first product container and the second product container therein. One non-limiting example of a suitable outer container is a carton. In this example, the carton is large enough to contain the first product container and the second product container within its confines. The carton may be made of any suitable material that provides the structural support for holding the first product container and the second product container. Materials useful as the carton include, without limitation, cardboard, paper, metal and plastic.

In an alternative embodiment, the outer container comprises shrink-wrap. In this embodiment, the first product container and the second product container are placed in close proximity with one another and shrink wrap is placed around the product containers, thereby physically binding the containers to each other.

The present packaging also optionally contemplates a divider being placed between the first product container and the second product container within the outer container. The divider physically separates the product containers, and may be present whether the outer container is a carton or shrink wrap. Materials useful as the divider include, without limitation, cardboard, paper, metal and plastic.

The outer container of the present packaging may also contain an item selected from the group consisting of a single bar code, a single new drug code and a single universal product code. Since the first product container and the second product container preferably contain distinct compositions, it is possible that the product containers may be separated from the outer container prior to purchase by a user, and the separate product containers may then be offered for individual sale. The presence of a single bar code, new drug code and/or universal product code on the outside of the outer container will help make the individual sale of the product containers more difficult. Additionally, in a preferred embodiment of the present packaging, both of the soap free cleanser and the topical composition are described on a single label and/or insert.

In a further embodiment of the present packaging, the first product container and the second product container are present in the outer container as unit dose containers. As unit dose containers, the first product containers and second product containers will each hold a single dose of the soap-free cleanser and topical composition, respectively. In this embodiment, the outer container will contain multiple first product containers and second product containers, each containing a single dose of the respective compositions.

The present subject matter also contemplates that the package will contain one or more additional product containers within the outer container. The one or more additional product containers preferably contain a composition distinct from the soap-free cleanser and the topical composition. In a preferred embodiment, the one or more additional product containers contain a barrier repair composition. Single dosage kits and packages containing once per day amounts of the present compositions may be prepared. Single dose, unit dose, and once-daily disposable containers of the mixtures and compositions herein are contemplated as within the scope of the present subject matter.

The present kits may comprise compositions which may be formulated for storage in a substantially non-reactive package. Storing the topical composition in a substantially non-reactive package may enhance stability of the product. This new method of storage provides enhanced product stability in comparison with the previous paper-based packages. Non-limiting examples of preferred non-reactive packages in this regard include a glass package, a molded or flexible plastic package, a single-dose vial, an aluminum package, a tin package, a composite cardboard package, a laminated package, a laminated pouch, a pump, and a combination thereof. Composite cardboard packages useful in this regard include wax coated cardboard packages.

In a preferred embodiment the substantially non-reactive package is a substantially non-reactive laminated or metal package. In a preferred embodiment, the metal may be coated. In an alternative preferred embodiment the metal is not coated. Additionally, in a preferred embodiment the composition for storage in the substantially non-reactive laminated or metal package is storage stable.

In preferred embodiments, the composition can be stored in the non-reactive package under a blanket of an inert gas. Preferred, non-limiting examples of inert gases useful in this regard include nitrogen gas, argon gas, and a mixture thereof.

The amount of composition per single packet may range from about 0.1 mL to about 20.0 mL, preferably between about 0.5 and about 5.0 mL, more preferably between about 1 and about 3 mL.

In another alternative embodiment, the present compositions can be administered using an applicator. Non-limiting examples of useful applicators in this regard include a pledget, a swab, a pad, and combinations thereof. Additionally, the present subject matter further contemplates that any of these topical compositions are provided in a package of less than 5 g topical composition as a unit of use.

The ability to formulate compositions capable of long term storage, without pre-mixing or compounding requirements prior to application, are also contemplated herein. Specifically, the present compositions remain unexpectedly stable in storage for periods including between about 2 weeks and about 18 months, preferably between about 3 weeks and about 15 months, more preferably between about 30 days and about 12 months.

Once-daily disposable packaging may also improve patient compliance, especially for teenagers.

### Methods of Use

The present subject matter also relates to a method for treating a skin disorder or condition in a patient, comprising:

topically administering a soap-free cleanser to skin of a patient in need thereof;

removing the soap-free cleanser from the skin;

topically administering to the patient a topical composition which comprises clindamycin or a pharmaceutically acceptable salt or ester thereof, and a pharmaceutically acceptable carrier in an amount effective to treat the skin disorder or condition.

Skin disorders or conditions treatable according to the present methods include but are not limited to microbial infections and inflammation of tissue. The microbial infections can be caused by gram-positive bacteria, gram-negative bacteria, and combinations thereof. Exemplary specific bacteria treatable by the present methods include but are not limited to P. acnes, Strep. pyogenes, E. coli, Pseudomonas aeruginosa, Staph. aureus, and combinations thereof.

Exemplary, non-limiting specific skin disorders, diseases, or conditions treatable by the present methods include but are not limited to acne, impetigo, rosacea, psoriasis, atopic dermatitis, secondary skin infections, responsive dermatoses, and combinations thereof. Other specific skin disorders treatable by the present methods include seborrhea, skin lesions, and bacterial skin infections. In a preferred embodiment, the skin disorder or condition improves following treatment with the present kits.

In a preferred embodiment, the patient to be treated is between the ages of 2 and 45. In a particularly preferred embodiment, the patient to be treated is between the ages of 10 and 35. In yet another preferred embodiment, the patient to be treated is between the ages of 12 and 25.

In a preferred embodiment, the soap-free cleanser is administered before the topical composition. In an alternative preferred embodiment the soap-free cleanser is administered after the topical composition. In another alternative preferred embodiment the soap-free cleanser is administered at the same time as the topical composition. In an additional preferred embodiment the soap-free cleanser is topically administered to and maintained in contact with the affected area for about 15 seconds to about 30 minutes before it is removed from the affected area. In another preferred embodiment the soap-free cleanser is topically administered to and maintained in contact with the affected area for about 15 seconds to about 120 seconds before it is removed from the affected area. Alternatively, in an additional preferred embodiment the soap-free cleanser is topically administered to and maintained in contact with the affected area for about 15 seconds to about 60 seconds before it is removed from the affected area.

### Reducing Irritation

Clindamycin-containing topical compositions can, at times, cause irritation of the affected area of skin to which it is applied. The irritation caused by clindamycin may be manifested by the appearance of redness in the skin following application of the clindamycin thereto. The irritation of the skin also may include drying out the skin, as well as slight pain.

Accordingly, in one aspect, the present subject matter is additionally directed to reducing this irritation associated with the application of clindamycin to the affected area. By reducing the irritation, the affected area of skin of the user will not exhibit as much redness often experienced by a user that undergoes a topical composition without the concomitant topical administration of a soap-free cleanser. Optimally, the present subject matter in this way will help to result in the elimination of the irritation resulting from the topical compositions.

In an alternate embodiment of the present subject matter, the soap-free cleanser is topically administered to an affected area before the topical composition is administered thereto. When the soap-free cleanser is administered before the topical composition, the soap-free cleanser removes reactive materials from the surface of the skin. The reactive materials include without limitation dead skin cells, dirt, excess sebum and microbes. If not removed by the soap-free cleanser, these reactive materials may interact with the topical clindamycin, thus contributing to the irritation associated with the topical composition. By removing the reactive materials prior to administering the topical composition, the reactive materials are no longer present to help cause such irritation.

In another embodiment of the present subject matter, the soap-free cleanser can be topically administered to an affected area after the topical composition is administered thereto. By administering the soap-free cleanser to the affected area following the topical composition thereof, the skin of the affected area is normalized and repair of any damage of the skin is enhanced. In this situation, the soap-free cleanser may act as a moisturizer of the skin to bring the skin back to its normal state. Thus, the irritation associated with the topical composition may be reduced as any dryness resulting from the topical composition is combated with the moisturizing properties of the soap-free cleanser.

In a further embodiment of the present subject matter, the soap-free cleanser can be topically applied to an affected area at the same time as the topical composition. By administering the soap-free cleanser at the same time as the topical composition, the potential irritating and oxidizing qualities of the topical composition may be diluted, resulting in reduced irritation of the affected area. Besides diluting the topical composition, the soap-free cleanser may also remove reactive materials from the surface of the skin prior to interaction with the topical composition.

Accordingly, the present subject matter may result in the reduction of irritation associated with a topical composition. The reduction of irritation may be measured by the absence of skin redness normally associated with a topical composition. The reduction of irritation may also be manifested by a lack of dryness of the affected area, as well as a lack of pain. In a particularly preferred embodiment, the irritation associated with the topical composition is completely reduced, meaning that no irritation occurs following administration of the topical composition to the affected area.

The soap-free cleanser is a short-contact therapy. As a short-contact therapy, it is preferable that the soap-free cleanser is removed from the affected area after a limited amount of time of contact. Preferably, the soap-free cleanser is removed using water.

In a non-limiting example, the soap-free cleanser is in contact with the affected area for the same amount of time, both compositions are in contact with the affected area for about 45 seconds or less before being removed.

### Enhancing Efficacy

The present subject matter is also directed to a method for enhancing the efficacy of a clindamycin-containing topical composition comprising:

a) topically administering a soap-free cleanser to an affected area; and

b) topically administering the clindamycin-containing topical composition to the affected area.

As discussed herein, acne is a condition of the human skin characterized by an excess flow of sebum, or skin oil, from the sebaceous glands located in the pilosebaceous apparatus. Clindamycin has been shown to be effective in the treatment of acne. In particular, clindamycin-containing topical compositions may be effective at reducing the number of comedones, inflammatory papules, pustules, cysts and lesions. Clindamycin-containing topical compositions may also be effective at reducing the microbial count associated with the secondary infection often formed in an acne-affected area of skin. However, the efficacy of clindamycin can be reduced by the presence of other reactive materials on the affected area of skin.

Accordingly, in another aspect, the present methods are further directed to enhancing the efficacy of a clindamycin-containing topical composition in treating acne. In this regard, the efficacy of the clindamycin-containing topical compositions is enhanced by topically administering a soap-free cleanser to the affected area of skin.

The present methods may enhance the efficacy of the clindamycin-containing topical composition, resulting in a reduction of the number of comedones, inflammatory papules, pustules, cysts and lesions. By including the step of topically administering a soap-free cleanser to the affected area, the number of comedones, inflammatory papules, pustules, cysts and lesions can typically be reduced by about 20% to about 80%. Preferably, the number of comedones, inflammatory papules, pustules, cysts and lesions can be reduced by about 40% to about 60%. More preferably, the number of comedones, inflammatory papules, pustules, cysts and lesions can be reduced by about 50%. The reduction in the number of comedones, inflammatory papules, pustules, cysts and lesions is observed when compared to the application of the clindamycin-containing topical composition without the concomitant administration of the soap-free cleanser.

The enhancement of the clindamycin-containing topical composition may also result in a reduction of the irritation caused by the clindamycin-containing topical composition. By reducing the irritation, the affected area of skin of the user will not exhibit as much redness as a user that undergoes treatment comprising topical administration of a clindamycin-containing composition without the subsequent topical administration of a soap-free cleanser. Optimally, the methods of the present subject matter will result in the elimination of any irritation resulting from the clindamycin-containing topical composition. The enhancement of the efficacy of the clindamycin-containing topical composition preferably results in from about 30% to about 70% reduction in the redness of the affected area. More preferably, the enhancement of the efficacy of the clindamycin-containing topical composition results in from about 40% to about 60% reduction in the redness of the affected area. Most preferably, the enhancement of the efficacy of the clindamycin-containing topical composition results in about 50% reduction of the redness of the affected area. The determination of the reduction in redness of the affected area is observed when compared to the application of the clindamycin-containing topical composition without the concomitant administration of the soap-free cleanser.

The enhancement of the efficacy of the clindamycin-containing topical composition may further result in a reduction of the microbial count in the affected area. As discussed herein, acne is caused by an interaction among hormones, keratin, sebum, and bacteria. Clindamycin may act as an anti-microbial agent to combat the bacteria and other microbes present in an area affected by acne. The step of topically administering a soap-free cleanser to an affected area enhances the efficacy of the anti-microbial activity of a clindamycin-containing topical composition. In addition to physically removing microbes and bacteria from the affected area, the soap-free cleanser removes organic material that the microbes and bacteria feed upon. In removing the organic material, the soap-free cleanser removes a source of food for the microbes, thereby keeping the microbes from proliferating. Further, the removal of the organic material, dirt, excess sebum and dead skin cells allows the clindamycin to come into direct contact with the microbes and hastens the oxidation of the microbes.

The enhancement of the efficacy of the clindamycin-containing topical composition as an anti-microbial material preferably result may result in a reduction of the microbial count by about 30% to about 70%. More preferably, the enhancement of the efficacy of the clindamycin-containing topical composition may result in a reduction of the microbial count by about 40% to about 60%. Most preferably, the enhancement of the efficacy of the clindamycin-containing topical composition may result in a reduction of the microbial count by about 50%. The reduction of the microbial count is observed when compared to the topical application of the clindamycin-containing composition without the concomitant administration of the soap-free cleanser.

The enhancement of the efficacy of the clindamycin-containing topical composition also may result in a faster return of the affected area to normal skin conditions, i.e., skin that is no longer affected by acne. Related to this result of the enhancement of the efficacy of the clindamycin-containing topical composition is that the appearance of the skin improves at a greater rate than observed without the concomitant administration of the soap-free cleanser. Acne manifests itself in the affected area by the appearance of redness, comedones, inflammatory papules, pustules, cysts and lesions. Likewise, the potentially irritating properties of topical clindamycin-containing composition may act on the skin to produce irritation in the form of redness, dryness and slight pain. The step of topically administering a soap-free cleanser in the present subject matter helps the skin to recover from the effects of acne, as well as the effects the clindamycin-containing topical composition, at a quicker rate than if the soap-free cleanser was not applied to the affected area.

Preferably, the enhancement of the efficacy of the clindamycin treatment results in a return of the affected area to its normal state and improvement in the appearance of the affected area within a time that is about one-half of the time required without the topical administration of the soap-free cleanser. More preferably, the time is about one-third of the time required without the topical administration of the soap-free cleanser. Most preferably, the time is about one-quarter of the time required without the topical administration of the soap-free cleanser.

In an alternate embodiment of the present subject matter, the soap-free cleanser is topically administered to an affected area before the topical composition is administered thereto. When the soap-free cleanser is administered before the topical composition, the soap-free cleanser removes reactive materials from the surface of the skin. The reactive materials include without limitation dead skin cells, dirt, excess sebum and microbes. By removing the reactive materials prior to administering the topical composition efficacy of the topical composition is increased.

In another embodiment of the present methods, the soap-free cleanser is topically administered to an affected area after the topical composition is administered thereto. As previously indicated, application of a clindamycin-containing topical composition to an affected area often results in irritation of the skin of the affected area. By administering the soap-free cleanser to the affected area following the clindamycin-containing topical composition thereof, the skin of the affected area is normalized and repair of any damage of the skin is enhanced. In this situation, the soap-free cleanser acts as a moisturizer of the skin to bring the skin back to its normal state thus enhancing the treatment of acne.

In a further embodiment of the present subject matter, the soap-free cleanser is topically applied to an affected area at the same time as the topical composition. By administering the soap-free cleanser at the same time as the topical composition, the irritating qualities of the clindamycin-containing topical composition, resulting in reduced irritation of the affected area. Besides diluting the clindamycin, the soap-free cleanser may also remove reactive materials from the surface of the skin prior to the oxidation of the skin by the clindamycin-containing topical composition, thus enhancing the efficacy of the clindamycin-containing topical composition as shown above.

### Methods of Production

The present subject matter is also directed to methods for making the soap-free cleanser and the topical composition.

Soap-Free cleanser

The soap-free cleanser of the present subject matter is preferably prepared by the following method:
1) Water is added to a suitable vessel;
2) The water is heated to a temperature between of about 72±2° C.;
3) While stirring, the emollient(s), surfactant(s) and preservatives are added to the vessel;
4) The resultant composition is stirred for about 20 minutes while maintaining the temperature at 72±2° C.;
5) A further emollient and further preservatives are added to a separate vessel;
6) Under continuous stirring, the components in the separate vessel are heated to 72±2° C. until all ingredients have melted and a uniform appearance is produced;
7) With stirring, the components of the separate vessel are slowly added to the first vessel;
8) Once added, the resulting composition is mixed for 10 minutes, then allowed to cool to 32±2° C.;
9) The cooled composition is packaged appropriately.

### Topical Compositions

A preferred topical composition of the present subject matter is preferably prepared by the following method:
1) Water is added to a first suitable vessel, e.g., the main kettle of a homogenizer, mill, or the like;
2) While string, the water is heated to a temperature between about 70±2° C.;
3) While stirring, the carbomer is slowly added to the heated water;
4) The resultant mixture is enclosed, a vacuum set at 200 Mbar is introduced, and the mixture is mixed for about 3 minutes;
5) The vacuum and mixing are stopped so the components of the homogenizer can be scraped down;
6) The lid is closed, the mixing restarted, and the vacuum pressure is reintroduced;
7) Maintaining a temperature of 70±2° C., the mixture is mixed for a period of ten minutes;
8) The mixing and vacuum is stopped and the lid is opened for the addition of surfactant(s), preservative(s), and Allantoin.
9) Once again, The lid is closed, the vacuum pressure is returned to 200 Mbar, and the mixture is mixed for 5 minutes.
10) Mixing the mixture for an additional 10 to 15 minutes, the vacuum pressure is adjusted to 400 Mbar, and the temperature is reduced to 30±2° C.;
11) As pH adjuster, Sodium Hydroxide Pellets are added to water in a second suitable vessel, under stirring, to create a Sodium Hydroxide Solution;
12) In the first suitable vessel, mixing and vacuum pressure are briefly stopped, the lid is opened, and the Sodium Hydroxide Solution is added to the mixture;
13) The lid is closed, mixing is restarted, the vacuum is at 300 Mbar, and the mixture is mixed for 15 minutes;
14) In a third separate suitable vessel, Clindamycin Phosphate is added to purified water;
15) The mixture is mixed for 5 minutes, or until clear.
16) Maintaining mixing in the first suitable vessel, the vacuum is set to 400 Mbar and a temperature of 30±2° C. is maintained;
17) The mixture of the third suitable vessel is added to the first suitable via a feed valve to allow for maintained mixing;
18) The mixture is mixed for 5 minutes;
19) Through a feed funnel, water is added to the mixture;
20) Maintaining mixing, the vacuum is increased to a setting of 500 Mbar, the temperature is maintained at 30±2° C., and the mixture is mixed for 10 minutes;
21) The mixture is placed into polyethylene or polyethylene lined drums;
22) The mixture, now the topical composition, may be properly packaged.

An alternative preferred topical composition of the present subject matter is preferably prepared by the following method:
1) Water is added to a suitable vessel;
2) Under string, Clindamycin Phosphate is added to the water and mixed for about 30 minutes, or until all powder is dissolved, to form a Clindamycin Phosphate solution;
3) Water is added to a second suitable vessel, e.g., a 40 gallon Groen Kettle, or the like, and under stirring conditions, the water is heated to 70±5° C.
4) Maintaining the temperature at 70±5° C., the carbomer(s) is added.
5) The mixing is stopped to allow for a scrape down of the side-wall of the vessel and mixer shafts to remove any product therefrom;
6) The mixing is restarted and maintained for 30 minutes at a temperature of 70±5° C.;
7) Mixing is again stopped, and the mixture is checked for unhydrated carbomer product.
8) Once the determination that no unhydrated carbomer product remains is made, mixing is restarted and maintained at a temperature of 70±5° C.;
9) Under stirring wetting agent(s), emulsifier(s), emollient(s), preservative(s), Silicon Dioxide, surfactant(s), sequestering agent(s), of Dow Fluid 200 is added to the mixture;
10) Under mixing, the temperature is cooled to 22.5±2.5° C. over a period of 0.5 to 3 hours;
11) The cooled mixture is mixed for a period of about ten minutes.
12) In yet an additional suitable vessel, under stirring, a pH modifier (Sodium Hydroxide, NF) is slowly added to water to form a Sodium Hydroxide solution;
13) The Sodium Hydroxide solution is added, under stirring conditions, to the mixture in the second suitable vessel while maintaining a batch temperature of 22.5±2.5° C.;
14) Mixing is continued for 10 minutes at a batch temperature of 22.5±2.5° C.;
15) The Clindamycin Phosphate solution from the first suitable vessel is slowly added to the mixture in the second suitable vessel under stirring conditions;
16) The resulting mixture is mixed for 10 minutes at a batch temperature of 22.5±2.5° C.;
17) While mixing, and maintaining a temperature of 22.5±2.5° C., 15.6 g of Titanium Dioxide, #3328 (Titanium Dioxide, USP) is added to the mixture.
18) Mixing is stopped so the side walls and mixer shafts may be scraped down of any product;
19) Any lumps of material in the mixture are broken-up with a clean, stainless steel paddle, spatula, or the like;
20) Mixing is commenced and maintained for 10 minutes at a batch temperature of 22.5±2.5° C.;
21) Again, mixing is stopped so the side walls and mixer shafts may be scraped down of any product;
22) Under agitated conditions, the mixture is processed through a Homogenizer, Mill, or the like, at a pressure setting of 2500±100 psi using a Rotary Lobe Pump, or the like;
23) A pressure between the Homogenizer, Mill, or the like, and the Rotary Lobe Pump, or the like is maintained between 40±20 psi;
24) Maintaining a batch temperature of 22.5±2.5° C., the product is recirculated back into the second suitable vessel and through the Homogenizer, Mill, or the like, until no particle exceeds 60 microns;
25) Maintaining a batch temperature of 22.5±2.5° C., once the appropriate particle size is obtained the, product is homogenized, milled, or the like, into polyethylene lined drums;
26) The product is poured into to a clean suitable vessel, for example, one similar or equivalent to the second suitable vessel, which is cool and at room temperature;
27) The product is mixed for ten minutes, maintaining a batch temperature of 22.5±7.5° C. The product is discharged directly into a polyethylene lined drum, or the like, using a Rotary Lobe Pump, or the like, set at about 8-20 Hz;
28) The product, now the topical composition, may be properly packaged.

### Routes of Administration/Dosage

To be effective, the route of administration for both the kit and the topical composition used in the present kit, package and methods must readily affect the affected area. In particular, acne is known to affect the face, neck, back, ears, and scalp. Moreover, it will be understood that this dosage of topical composition can be administered in a single or multiple dosage units to provide the desired therapeutic effect.

The preferred topical composition may be administered in a single or multiple doses daily. The preferred topical composition may also be administered according to a schedule in which one of the cleanser and the topical composition is administered in the morning, while the other of the cleanser and the topical composition is administered at night. Further, as previously indicated, the present methods contemplate the administration of the soap-free cleanser before the topical composition, at the same time as the topical composition, and after the topical composition. In a preferred embodiment, the topical compositions are administered from one to three times daily, preferably with a concomitant administration of the soap-free cleanser. Starting with a low dose twice daily and slowly working up to higher doses if needed is a preferred strategy. The amount of active ingredients that may be combined with the carrier materials to produce an appropriate dosing regimen form will vary depending upon the host treated, the nature of the disease, disorder, or condition, and the nature of the active ingredient(s).

Dosage levels for the active ingredients disclosed herein are well known in the art and are selected to maximize the treatment of the above conditions. The specific dose level for any particular patient will vary depending upon a variety of factors, including the activity of the specific compound employed; the age, body weight, general health, sex and diet of the patient; the time of administration; the rate of excretion; drug combination; the severity of the particular disease being treated; and the form of administration. Typically, in vitro dosage-effect results can provide useful guidance on the proper doses for patient administration. Studies in animal models are also helpful. The considerations for determining the proper dose levels are well known in the art and are incorporated herein for the present subject matter.

The optimal pharmaceutical formulations will be determined by one skilled in the art depending upon considerations such as the particular drug or drug combination and the desired dosage. See, for example, “Remington's Pharmaceutical Sciences”, 18th ed. (1990, Mack Publishing Co., Easton, Pa. 18042), pp. 1435-1712, the disclosure of which is hereby incorporated by reference in its entirety. Such formulations may influence the physical state, stability, rate of in vivo release, and rate of in vivo clearance of the therapeutic agents. Pharmacokinetic parameters such as bioavailability, absorption rate constant, apparent volume of distribution, unbound fraction, total clearance, fraction excreted unchanged, first-pass metabolism, elimination rate constant, half-life, and mean residence time are well known in the art.

Lessening exposure by once-daily administration affects multiple pharmacokinetic parameters and provides the initial mechanism for avoiding skin irritation and inflammation and the other toxicity issues discussed herein. Additional formulations may be prepared which factor in the benefit/risk ratio for a clindamycin composition. The level of toxicity of these compounds is known and reference is made to the package inserts for Cleocin T® and the level of adverse events reported from their clinical trials.

### EXAMPLES

The following examples are illustrative of preferred soap-free cleanser and topical compositions and are not intended to be limitations thereon. All polymer molecular weights are mean average molecular weights. All percentages are based on the percent by weight of the final formulation prepared unless otherwise indicated and all totals equal 100% by weight.

In the examples of topical compositions disclosed herein, the following ingredients are used: carbomers and polymeric emulsifiers (polyacrylates), such as for example Carbopol® 940 from Noveon Inc., Cleveland, Ohio; disodium monolauryl sulfosuccinate, such as Monamate LA-100; emulsifier-solubilizer-stabilizers block PEG/PPG co-polymers such as Poloxamer 182, also known as Pluracare® L-62; surfactant-emollient-lubricant-plasticizers such as dimethicone also known as Dow Fluid 200; sequestering agents such as disodium EDTA, commercially known as Hampene® Na₂; and hydrated silica and absorbants, such as Syloid 244 FP.

### Example 1

The following example illustrates the preparation of a preferred soap-free cleanser of the present subject matter. The soap-free cleanser comprises:

| | % W/W |
|---|---|
| Purified Water | 91.50 |
| Polyethylene Glycol 3350NF | 5.00 |
| Sodium Cocoyl Isethionate | 0.70 |
| Methylparaben, NF | 0.20 |
| Stearyl Alcohol | 2.50 |
| Propylparaben, NF | 0.05 |
| Butylparaben, NF | 0.05 |
| | 100.0% |

The soap-free cleanser was prepared by adding 3020 kg of purified water to a suitable vessel. The water was heated under stirring to a temperature of 72±2° C. within 55 to 180 minutes. As the stirring continued, 165 kg of polyethylene glycol 3350, NF, 23.1 kg of sodium cocoly isethionate, and 6.6 kg of methylparaben, NF were added to the heated water. The mixture was mixed for 20 minutes while maintaining the temperature. In a separate vessel, 82.5 kg of stearyl alcohol, 1.65 kg of propylparaben and 1.65 kg of butyl paraben are mixed and heated to 72±2° C. within 45 to 85 minutes until all ingredients have melted and a uniform appearance is produced. While continually stirring, the mixture from the second vessel is slowly added to the mixture of the first vessel. The combined mixtures are stirred for 10 minutes while the temperature is maintained at 72±2° C. While continuous stirring, the composition is cooled to a temperature of 32±2° C. within 135 to 155 minutes. The cooled composition is then properly packaged.

### Example 2

The following example illustrates the preparation of a preferred soap-free cleanser of the present subject matter. The soap-free cleanser was prepared in accordance with the method as set forth in Example 1. The soap-free cleanser comprises:

| | % W/W |
|---|---|
| Purified Water | 91.75 |
| Polyethylene Glycol 75 | 5.00 |
| Sodium Cocoyl Isethionate | 0.45 |
| Methylparaben, NF | 0.20 |
| Stearyl Alcohol | 2.50 |
| Propylparaben, NF | 0.05 |
| Butylparaben, NF | 0.05 |
| | 100.0% |

### Example 3

The following example illustrates the preparation of a preferred soap-free cleanser of the present subject matter. The soap-free cleanser was prepared in accordance with the method as set forth in Example 1. The soap-free cleanser comprises:

| | % W/W |
|---|---|
| Purified Water | 90.70 |
| Polyethylene Glycol 3350 | 5.00 |
| Sodium Cocoyl Isethionate | 1.50 |
| Methylparaben, NF | 0.20 |
| Stearyl Alcohol | 2.50 |
| Propylparaben, NF | 0.05 |
| Butylparaben, NF | 0.05 |
| | 100.0% |

### Example 4

The following example illustrates the preparation of a preferred soap-free cleanser of the present subject matter. The soap-free cleanser was prepared in accordance with the method as set forth in Example 1. The soap-free cleanser comprises:

| | % W/W |
|---|---|
| Purified Water | 91.68 |
| Polyethylene Glycol 3350 | 5.00 |
| Sodium Cocoyl Isethionate | 0.70 |
| Methylparaben, NF | 0.40 |
| Stearyl Alcohol, NF (Crdacol S-95, NF) | 2.50 |
| Propylparaben, NF | 0.04 |
| Butylparaben, NF | 0.04 |
| | 100.0% |

### Example 5

The following example illustrates the preparation of a preferred topical composition of the present subject matter. The topical composition comprises:

| | % W/W |
|---|---|
| Clindamycin Phosphate | 1.29 |
| Purified Water | 83.76 |
| Carbopol 934P | 1.00 |
| Methylparaben | 0.30 |
| Allantoin | 0.50 |
| Carbowax 400 | 8.00 |
| Propylene Glycol | 5.00 |
| Sodium Hydroxide | 0.15 |
| | 100.0% |

The topical composition is prepared by adding 33.3 kg of purified water to the main kettle of a homogenizer. While string, the water is heated to a temperature of 70±2° C. As the stirring continues, the lid of the kettle is slowly opened and 500 g of Carbopol 934P is slowly added to the heated water. The lid is closed and the mixture is mixed for 3 minutes. A vacuum pressure is set at 200 Mbar. The vacuum and mixing are stopped so the components of the homogenizer can be scraped down. The lid is closed, the mixing restarted, and the vacuum pressure is reintroduced. Maintaining a temperature of 70±2° C., the mixture is mixed for a period of ten minutes. Once again, the mixing and vacuum pressure is stopped and the lid is opened. To the mixture, 150 g of Methylparaben, 250 g of Allantoin, 4.0 kg of Carbowax, and 2.50 kg of Propylene Glycol, are added. The lid is closed, the vacuum pressure is returned to 200 Mbar, and the mixture is mixed for 5 minutes. While continuing to mix the mixture for an additional 10 to 15 minutes, the vacuum pressure is adjusted to 400 Mbar and the temperature is reduced to 30±2° C.

In a second suitable vessel, 75.0 g of Sodium Hydroxide Pellets are added. Under stirring, 1.04 kg of Purified Water is slowly introduced to the Sodium Hydroxide Pellets until the Sodium Hydroxide Pellets are dissolved to create a Sodium Hydroxide Solution.

In the kettle, mixing and vacuum pressure are briefly stopped and the lid is opened. The Sodium Hydroxide Solution is added to the mixture. The lid is closed, mixing is restarted, and the vacuum is at 300 Mbar. The mixture is mixed for 15 minutes.

In a third separate suitable vessel, 7.5 kg of purified water is added. To the vessel containing the Purified Water, 0.647 kg of Clindamycin Phosphate is added. The mixture is mixed for 5 minutes or until clear.

Maintaining mixing in the kettle, the vacuum is set to 400 Mbar; a temperature of 30±2° C. is maintained. The mixture of the third suitable vessel is added to the kettle via a feed valve to allow for maintained mixing. The mixture is mixed for 5 minutes. Through a feed funnel, 0.040 kg of Purified Water is added to the mixture. Maintaining mixing, the vacuum is increased to a setting of 500 Mbar. The temperature is maintained at 30±2° C. and the mixture is mixed for 10 minutes. Finally, the mixture is placed into polyethylene or polyethylene lined drums. The mixture, now the topical composition, may be properly packaged.

### Example 6

The following example illustrates the preparation of an alternative preferred topical composition of the present subject matter. The topical composition comprises:

| | % W/W |
|---|---|
| Purified Water, USP | 91.11 |
| Carbopol 980 (Carbomer 940, NF) | 2.50 |
| Poloxamer 182 (Pluronic L62) | 0.00 |
| Glycerin, USP (96%) | 4.00 |
| Methylparaben, NF | 0.10 |
| Silicon Dioxide, NF (Syloid 244FP) | 0.25 |
| Disodium Lauryl Sulfosuccinate | 0.04 |
| (Monamate LA-100) | |
| Edetate Disodium, USP (Hampene Na₂) | 0.10 |
| Dow Fluid 200 (100 CS) | 0.10 |
| Clindamycin Phosphate | 1.28 |
| Titanium Dioxide #3328 | 0.01 |
| (Titanium Dioxide, USP) | |
| | 100.0% |

The topical composition is prepared by adding 29.5 kg of Purified Water, USP to a suitable vessel. While string, 1.499 kg of Clindamycin Phosphate is added to the water. The mixture is mixed for 30 minutes or until all powder is dissolved to form a Clindamycin Phosphate solution. The mixing should not exceed 60 minutes.

To a second suitable vessel, for example, a 40 gallon Groen Kettle, or the like, 77.9 kg of Purified Water, USP is added. Under stirring conditions, the water is heated to 70±5° C. Maintaining the temperature at 70±5° C., 3.0 kg of Carbopol 980 (Carbomer 940, NF) is added. The mixing is stopped to allow for a scrape down of the side-wall of the vessel and mixer shafts to remove any product. The mixing is restarted and maintained for 30 minutes at a temperature of 70±5° C. Mixing is again stopped and the mixture is checked fro unhydrated Carbopol product. Once the determination that no unhydrated Carbopol product remains is made, mixing is restarted and maintained at a temperature of 70±5° C. The following is added to the stirring mixture: (1) 240.0 g of Poloxamer 182 (Pluronic L62); (2) 4.8 kg of Glycerin, USP (96%); 120 g of Methylparaben, NF; (4) 300.0 g of Silicon Dioxide, NF (Syloid 244FP); (5) 48.0 g of Disodium Lauryl Sulfosuccinate (Monamate LA-100); (6) 120.0 g of Edetate Disodium, USP (Hampene Na₂); and (7) 120 g of Dow Fluid 200 (100 CS). Continuing mixing of the mixture over a period of 0.5 to 3 hours, the temperature is cooled to a temperature of 22.5±2.5° C., the mixture is mixed for a period of ten minutes.

In yet an additional suitable vessel, under stirring conditions, 372 g of Sodium Hydroxide, NF is slowly added to 1.40 kg of Purified Water, USP to form a Sodium Hydroxide solution.

The Sodium Hydroxide solution is added, under stirring conditions, to the mixture in the second suitable vessel while maintaining a batch temperature of 22.5±2.5° C. Mixing is continued for 10 minutes at a batch temperature of 22.5±2.5° C.

The Clindamycin Phosphate solution from the first suitable vessel is slowly added to the mixture in the second suitable vessel under stirring conditions. The resulting mixture is mixed for 10 minutes at a batch temperature of 22.5±2.5° C. While mixing, and maintaining a temperature of 22.5±2.5° C., 15.6 g of Titanium Dioxide, #3328 (Titanium Dioxide, USP) is added to the mixture. Mixing is stopped so the side walls and mixer shafts may be scraped down of any product. Any lumps of material in the mixture are broken-up with a clean, stainless steel paddle, spatula, or the like. Mixing is commenced and maintained for 10 minutes at a batch temperature of 22.5±2.5° C. Again, mixing is stopped so the side walls and mixer shafts may be scraped down of any product. Under agitated conditions, the mixture is processed through a Homogenizer, Mill, or the like, at a pressure setting of 2500±100 psi using a Rotary Lobe Pump, or the like. A pressure between the Homogenizer, Mill, or the like, and the Rotary Lobe Pump, or the like is maintained between 40±20 psi. Maintaining a batch temperature of 22.5±2.5° C., the product is re-circulated back into the second suitable vessel and through the Homogenizer, Mill, or the like, until no particle exceeds 60 microns. The particle size may be determined by, for example, a Hegman Guage, or the like. Maintaining a batch temperature of 22.5±2.5° C., once the appropriate particle size is obtained the, product is homogenized, milled, or the like, into polyethylene lined drums.

The product is poured into to a clean suitable vessel, for example, one similar or equivalent to the second suitable vessel, which is cool and at room temperature. The product is mixed for ten minutes, maintaining a batch temperature of 22.5±7.5° C. The product is discharged directly into a polyethylene lined drum, or the like, using a Rotary Lobe Pump, or the like, set at 8-20 Hz. The product, now the topical composition, may be properly packaged.

Dow Fluid 200, or DOW CORNING 200® FLUID, 100 CS., is a 100% active, 00 cSt, polydimethylsiloxane polymer.

### Example 7

A highly stable gel composition is prepared using the following components. The active ingredients are benzoyl peroxide and clindamycin phosphate. The remaining components are inert or auxiliary.

| | |
|---|---|
| Ingredient | Parts by Weight |

Gel:

| | |
|---|---|
| Purified Water | 86.50% |
| Carbomer | 2.00% |
| Disodium monolauryl sulfosuccinate | 0.04% |
| Disodium EDTA | 0.10% |
| Methylparaben | 0.30% |
| Total: | 88.94% |

The gel is combined with the following to produce the instant
composition:
Wetting Agents and Emollients:

| | |
|---|---|
| Poloxamer 182 | 0.20% |
| Glycerin | 4.00% |
| Dimethicone | 0.10% |
| Hydrated Silica | 0.25% |
| Total: | 4.55% |

pH Adjustment:

| | |
|---|---|
| Sodium Hydroxide | 0.31% |
| Total: | 0.31% |

Active Ingredients:

| | |
|---|---|
| Benzoyl Peroxide | 5.00% |
| Clindamycin Phosphate | 1.20% |
| Total: | 6.20% |
| Total for Composition: | 100.00% |

### Example 8

The following topical composition is obtained when the following component formulations are mixed in equal parts, and later combined to yield the highly stable product.

| | |
|---|---|
| Ingredient | Parts by Weight |

Gel:

| | |
|---|---|
| Purified Water | 82.70% |
| Carbomer | 2.00% |
| Disodium monolauryl sulfosuccinate | 0.04% |
| Disodium EDTA | 0.10% |
| Methylparaben | 0.12% |
| Total: | 85.14% |

The gel is combined with the following to produce the instant
composition:
Wetting Agents and Emollients:

| | |
|---|---|
| Poloxamer 182 | 0.20% |
| Glycerin | 4.00% |
| Dimethicone | 0.10% |
| Hydrated silica | 0.25% |
| Total: | 4.55% |

pH Adjustment:

| | |
|---|---|
| Sodium Hydroxide | 0.31% |
| Total: | 0.31% |

Active Ingredients:

| | |
|---|---|
| Benzoyl Peroxide | 10.00% |
| Clindamycin Phosphate | — |
| Total: | 10.00% |
| Total for Composition: | 100.00% |

| | |
|---|---|
| Ingredient | Parts by Weight |

Gel:

| | |
|---|---|
| Purified Water | 90.30% |
| Carbomer | 2.00% |
| Disodium monolauryl sulfosuccinate | 0.04% |
| Disodium EDTA | 0.10% |
| Methylparaben | 0.30% |
| Total: | 92.74% |

The gel is combined with the following to produce the instant
composition:
Wetting Agents and Emollients:

| | |
|---|---|
| Poloxamer 182 | 0.20% |
| Glycerin | 4.00% |
| Dimethicone | 0.10% |
| Hydrated silica | 0.25% |
| Total: | 4.55% |

pH Adjustment:

| | |
|---|---|
| Sodium Hydroxide | 0.31% |
| Total: | 0.31% |

Active Ingredients:

| | |
|---|---|
| Benzoyl Peroxide | — |
| Clindamycin Phosphate | 2.40% |
| Total: | 2.40% |
| Total for Composition: | 100.00% |

The resultant mixture is essentially 10% of benzoyl peroxide with essentially 2% clindamycin.

### Example 9

A patient is suffering from acne. The soap-free cleanser of Example 4 is topically administered to the patient, followed by topical administration of the topical composition of Example 5. It would be expected that the patient would improve his/her condition or recover.

### Example 10

A patient is suffering from acne. The topical composition of Example 5 is topically administered to the patient, followed by topical administration of the soap-free cleanser of Example 2. It would be expected that the patient would improve his/her condition or recover.

### Example 11

A patient is suffering from a skin condition wherein the microbe is involved in a primary or secondary symptom. The topical composition of Example 5 is topically administered to the patient, followed by topical administration of the soap-free cleanser of Example 2. It would be expected that the patient would improve his/her condition or recover. (see paragraph 116)

### Example 12

A patient is suffering from a skin condition wherein the skin exhibit inflammation from a primary or secondary infection caused by a microbe. The topical composition of Example 5 is topically administered to the patient, followed by topical administration of the soap-free cleanser of Example 2. It would be expected that the patient would improve his/her condition or recover.

The present subject matter being thus described, it will be apparent that the same may be modified or varied in many ways. Such modifications and variations are not to be regarded as a departure from the spirit and scope of the present subject matter, and all such modifications and variations are intended to be included within the scope of the following claims.

We claim:

1. A kit, comprising:
a soap-free cleanser; and
a topical composition which comprises clindamycin or a pharmaceutically acceptable salt or ester thereof, and a pharmaceutically acceptable carrier.

2. The kit according to claim 1, wherein the clindamycin comprises about 0.9% to about 2.5% by weight of the total topical composition.

3. The kit according to claim 1, wherein the topical composition has a single clindamycin degradate of not more than about 0.02% by weight.

4. The kit according to claim 1, wherein the soap-free cleanser comprises water, at least one emollient, a surfactant and at least one preservative.

5. The kit according to claim 1, wherein the soap-free cleanser comprises
a) water in an amount of from about 85% by weight to about 95% by weight of the soap-free cleanser;
b) at least one emollient selected from the group consisting of polyethylene glycol, stearyl alcohol, and mixtures thereof and present in an amount of from about 5% by weight to about 10% by weight of the soap-free cleanser;
c) a surfactant selected from the group consisting of betaines, amine oxides, amphoteric surfactants, sulfates, isethionates, sulfoacetates, sarcosinates, phosphates, and mixtures thereof and present in an amount of from about 0.25% by weight to about 2% by weight of the soap-free cleanser; and
d) at least one preservative and present in an amount of about 0.1% by weight to about 0.5% by weight of the soap-free cleanser.

6. The kit according to claim 1, wherein the topical composition is selected from the group consisting of a gel, cream, lotion, suspension, emulsion, ointment, foam, and mixtures thereof.

7. The kit according to claim 1, wherein the pharmaceutically acceptable carrier comprises an inactive ingredient selected from the group consisting of carbomer, disodium monolauryl sulfosuccinate, disodium EDTA, methyl paraben, poloxamer, glycerin, dimethicone, hydrated silica, sodium hydroxide, purified water, and mixtures thereof.

8. The kit according to claim 1, wherein the topical composition has a final pH of about 4.6 to about 4.8.

9. The kit according to claim 1, wherein the topical composition further comprises at least one additional ingredient selected from the group consisting of benzoyl peroxide, salicylic acid, a retinoid, derivatives thereof, and mixtures thereof.

10. The kit according to claim 9, wherein the benzoyl peroxide dispersion comprises from about 2.25% to about 12.5% by weight of the total topical composition.

11. A package comprising:
an outer container; and
a first product container and a second product container within the outer container,
wherein the first product container contains a soap-free cleanser; and
the second product container contains a topical composition which comprises clindamycin or a pharmaceutically acceptable salt or ester thereof, and a pharmaceutically acceptable carrier.

12. The package according to claim 11, wherein the clindamycin comprises about 0.9% to about 2.5% by weight of the total topical composition.

13. The package according to claim 11, wherein the topical composition has a single clindamycin degradate of not more than about 0.02% by weight.

14. The package according to claim 11, wherein the soap-free cleanser comprises
a) water in an amount of from about 85% by weight to about 95% by weight of the soap-free cleanser;
b) at least one emollient selected from the group consisting of polyethylene glycol, stearyl alcohol, and mixtures thereof and present in an amount of from about 5% by weight to about 10% by weight of the soap-free cleanser;
c) a surfactant selected from the group consisting of betaines, amine oxides, amphoteric surfactants, sulfates, isethionates, sulfoacetates, sarcosinates, phosphates, and mixtures thereof and present in an amount of from about 0.25% by weight to about 2% by weight of the soap-free cleanser; and
d) at least one preservative and present in an amount of about 0.1% by weight to about 0.5% by weight of the soap-free cleanser.

15. The package according to claim 11, wherein the topical composition is selected from the group consisting of a gel, cream, lotion, suspension, emulsion, ointment, foam, and mixtures thereof.

16. The package according to claim 11, wherein the topical composition further comprises inactive ingredients selected from the group consisting of carbomer, disodium monolauryl sulfosuccinate, disodium EDTA, methyl paraben, poloxamer, glycerin, dimethicone, hydrated silica, sodium hydroxide, purified water, and mixtures thereof.

17. The package according to claim 11, wherein the second product container is a substantially non-reactive laminated or metal package to enhance stability of the package.

18. The package according to claim 11, wherein the outer container is a carton.

19. The package according to claim 11, wherein the outer container is shrink-wrap.

20. The package according to claim 11, further comprising a divider between said first container and said second container.

21. The package according to claim 11, wherein the outer container contains an item selected from the group consisting of a single bar code, a single new drug code and a single universal product code.

22. The package according to claim 11, wherein the package further comprises one or more additional product containers within the outer container.

23. The package according to claim 11, wherein said first container and said second container are unit dose containers.

24. The package according to claim 11, wherein both of said soap free cleanser and said topical composition are described on a single label and/or insert.

25. A method for treating a skin disorder or condition in a patient, comprising:
topically administering a soap-free cleanser to skin of a patient in need thereof;
removing the soap-free cleanser from the skin;
topically administering to the patient a topical composition which comprises clindamycin or a pharmaceutically acceptable salt or ester thereof, and a pharmaceutically acceptable carrier in an amount effective to treat the skin disorder or condition.

26. The method according to claim 25, wherein administration of the soap-free cleanser comprises administration selected from the group consisting of administration before the topical composition, administration after the topical composition, and administration at the same time as the topical composition.

27. The method according to claim 25, wherein the soap-free cleanser is topically administered to and maintained in contact with an affected area for a time period selected from the group consisting of about 15 seconds to about 30 minutes before it is removed from the affected area, about 15 seconds to about 120 seconds before it is removed from the affected area, and about 15 seconds to about 60 seconds before it is removed from the affected area.

28. The method according to claim 25, wherein said skin condition or disorder is a microorganism or microbial infection susceptible to topical treatment thereof.

29. The method according to claim 25, wherein said skin disorder or condition includes a microbial infection.

30. The method according to claim 29, wherein said microbial infection is caused by bacteria selected from the group consisting of gram-positive bacteria, gram-negative bacteria, and combinations thereof.

31. The method according to claim 30, wherein said bacteria is selected from the group consisting of P. acnes, Strep. Pyogenes, E. coli, Pseudonomas originosa, Staph. Aureus, and mixtures thereof.

32. The method according to claim 31, wherein said skin disorder or condition includes an inflammation of tissue.

33. The method of claim 32, wherein said skin disorder is selected from the group consisting of acne, impetigo, rosacea, psoriasis, atopic dermatitis, secondary skin infections, responsive dermatoses, and combinations thereof.
